(19) United States
(12) Patent Application Publication
(10) Pub. No.: US 2022/0220686 A1
(43) Pub. Date: Jul. 14, 2022

(54) HYDROSTATICALLY COMPENSATED DEVICE FOR GROUND PENETRATION RESISTANCE MEASUREMENTS

(71) Applicant: KING ABDULLAH UNIVERSITY OF SCIENCE AND TECHNOLOGY, Thuwal (SA)

(72) Inventors: Marco TERZARIOL, Thuwal (SA); Juan Carlos SANTAMARINA, Thuwal (SA)

(21) Appl. No.: 17/604,853

(22) PCT Filed: Mar. 26, 2020
(86) PCT No.: PCT/IB2020/052873

§ 371 Date: Oct. 19, 2021

Related U.S. Application Data
(60) Provisional application No. 62/839,224, filed on Apr. 26, 2019.

(51) Int. Cl. E02D 1/02; G01N 3/42; E02D 1/06

(52) U.S. Cl. CPC E02D 1/022; G01N 33/24; E02D 1/06; G01N 3/42

(57) ABSTRACT

A device for measuring ground penetration resistance that can perform this type of measurements with a hydrostatically compensated tip. Measurements of penetration resistance use a hydraulic compensated cone, which addresses the issue of very soft sediments at high water depths. The cone includes a floating tip, a body, a housing and a sleeve. Low viscosity oil fills the internal cavity between the tip and the core. A differential pressure transducer located at the top of the cone body determines the difference between the insertion related pressure and the background water pressure. This system compensates for this pressure difference using the pore fluid during insertion instead of the water column, which results in greater accuracy.

### CROSS-REFERENCE TO RELATED APPLICATIONS

This application claims priority to U.S. Provisional Patent Application No. 62/839,224, filed on Apr. 26, 2019, entitled “HYDROSTATICALLY COMPENSATED PROBE FOR SOIL PENETRATION RESISTANCE IN LABORATORY AND FIELD MEASUREMENTS,” the disclosure of which is incorporated herein by reference in its entirety.

### BACKGROUND

### Technical Field

Embodiments of the subject matter disclosed herein generally relate to a system and method for measuring ground penetration resistance, and more particularly, to a device that can perform these types of measurement with a hydrostatically compensated tip.

### Discussion of the Background

The need for new energy sources has pushed oil and gas exploration to deeper waters, in what is known as offshore oil and gas exploration. To build the necessary oil infrastructure at these deep sea locations, for example, on the bottom of the ocean, which may be about 1,000 to 6,000 m deep, there is a growing need for high quality, in-situ testing, of the seabed soft sediments. The most common tool for characterization of these fine sediments is a cone that penetrates the ocean bottom and the test associated with it is called the cone penetration test (CPT). In addition, it is possible to use full-flow penetrometers such as the T-bar and Ball-cone.

A traditional tool to perform the CPT test is shown in FIGS. 1 and 2. FIG. 1 shows the entire tool 10 having a large robust member 14 that holds in its tip 16, a secondary probe 18. The secondary probe 18 holds, as shown in FIG. 2, a miniature pressure sensor 20. The large robust member 14 holds a pressure chamber 38 filled with oil 55, through a connecting tube 54. The connecting tube 54 communicates with a pump (not shown) located at the surface. The pump supplies the oil under pressure to force the probe member 18 to enter into the soil, when the primary probe 14 lands on the ocean bottom. A rod 60 extends from the probe member 18 into the pressure chamber 38 and guides the probe member 18 into the soil.

However, such a system is cumbersome and not reliable at high-water depths. A serious concern has risen during the last decades with respect to the test at the seabed at high-water depths because the standard push cones are not hydrostatically compensated for the water pressure. As the water depth at the testing location can get up to 6,000 meters, a standard cone having a surface of 10 cm² would experience a force of 60 kN (6,000 kg). The soft sediments resistant force on the same cone at the seabed level can be as low as 0.01 kN (1 kg), which represents <0.02% with respect to the hydrostatic water pressure at that level. The current standard push cones cannot measure with such resolution and accuracy in the presence of the high hydrostatic water pressure. At the same time, as the penetrometer deepens in the sediment, the local water pressure will increase and result in less reliable readings.

Returning to the oil filled push cone shown in FIGS. 1 and 2, it has a standard cone partially compensated for the water column. However, this device is not reliable at high-water depths. Over the past number of years, different solutions have been proposed. For example, full-flow penetrometers that are partially water-compensated, have an external reduction of their shaft, which make them internally hydrostatically stressed. Further, such devices have a specially designed notch and strain gauge that can read the penetration forces rather than the impact of the water pressure on the cone. However, even these devices do not fully remove the negative influence of the hydrostatic water pressure on the measurements.

Thus, there is a need for a new tool that is simple and is not negatively affected by the high hydrostatic water pressure at large measuring depths.

### BRIEF SUMMARY OF THE INVENTION

According to an embodiment, there is a push cone device for measuring a penetration resistance into ground. The device includes a body having an internal chamber, electronics located in the internal chamber, a sensing module attached to the body and configured to house one or more sensors, a tip resistance module attached to the sensing module and having a tip that is configured to be fully hydrostatically-balanced under water, and a differential pressure sensor that measures the penetration resistivity experienced by the tip resistance module.

According to another embodiment, there is a push cone device for measuring a penetration resistivity into ground. The device includes a body having an internal chamber that houses electronics, and a tip attached to the body and being configured to move relative to the body. The tip is configured to be pressure balanced under water.

According to yet another embodiment, there is a method for measuring a penetration resistance with a push cone device. The method includes a step of lowering the push cone device to the ocean bottom, a step of self-balancing a hydrostatic water pressure acting on a tip of the push cone device so that a net pressure on the tip is negligible, a step of pushing the tip into the ground, and a step of measuring with a differential pressure sensor a pressure associated with the penetration resistance generated by the ground against the tip. The differential pressure sensor is configured to be in fluid communication, at a first port, with an oil chamber located inside the push cone device and, at a second port, with a water passage also inside the push cone.

### BRIEF DESCRIPTION OF THE DRAWINGS

For a more complete understanding of the present invention, reference is now made to the following descriptions taken in conjunction with the accompanying drawings, in which:

FIGS. 1 and 2 illustrate a conventional push cone device;

FIG. 3 illustrates a push cone device that has a balanced hydrostatic water pressure tip resistance module;

FIG. 4 illustrates a detail of the hydrostatically balanced tip resistance module;

FIG. 5 illustrates a detail of a tip of the tip resistance module;

FIG. 6 illustrates the push cone device when pushed into the ground;

FIGS. 7A and 7B illustrate various ways for deploying the push cone device into the ground;

FIGS. 8A, 8B and 8C illustrate various parameters measured with the push cone device when deployed into the ground; and

FIG. 9 is a flowchart of a method for measuring a penetration resistance with the novel push cone device.

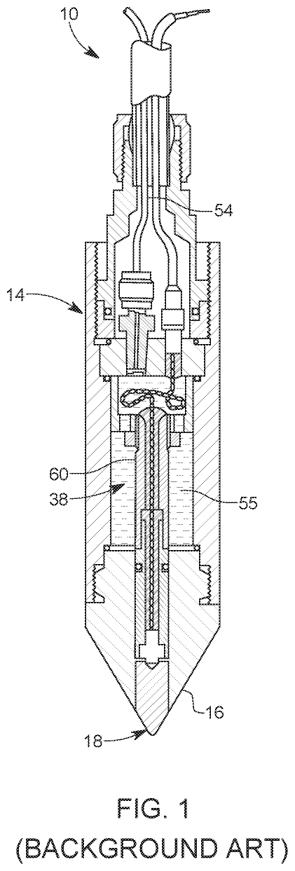

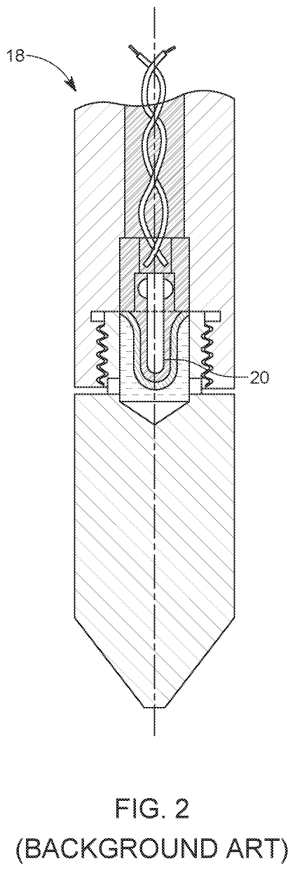

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a system that uses a differential pressure transducer that factors out the influence of the hydrostatic pressure of the water. However, the embodiments to be discussed next are not limited to such a transducer, but may be used with other sensors that achieve the same result.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a stackable, modular, push cone device is configured to balance out the hydrostatic water pressure acting on the tip of the device and uses a differential pressure transducer to read a pressure acting on the tip of the device due to a resistance generated by the ground (soil) when the tip penetrates the ground. One or more advantages associated with this device are: (1) the tip of the pushing cone is fully compensated for the hydrostatic water pressure; (2) measurements performed with this device are not in the hydrostatically stressed shaft; and (3) more accurate and higher resolution measurements (for example, a resolution of +/−2.5 kPa of the penetration resistance under a 70 MPa of hydrostatic water pressure) can be obtained when compared to the current systems.

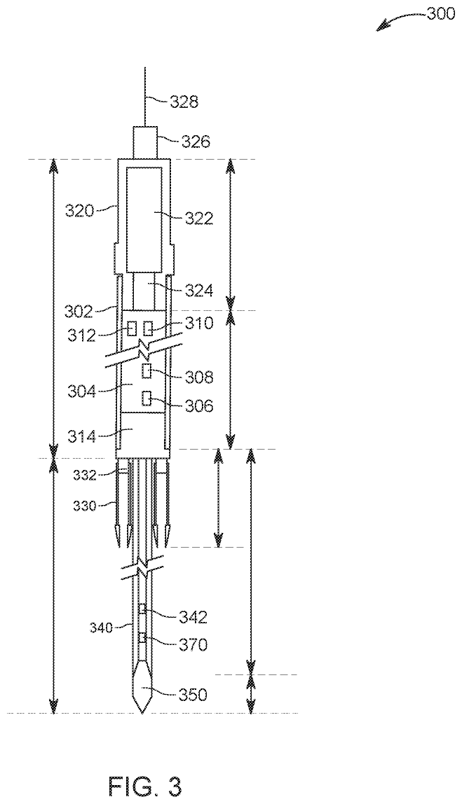

As illustrated in the embodiment of FIG. 3, the novel push cone device 300 has a body 302 that defines an internal housing 304, which is sealed from the ambient environment, so that seawater cannot enter. The internal housing 304 may be configured to host a processor 306, a memory 308 for storing data recorded by the various sensors associated with the device 300, an optional power source 310, for example, a battery, and sensor electronics 312. The sensor electronics 312, for example, analog-to-digital converters, amplifiers, etc., may be configured to provide support for a temperature sensor, a water pressure sensor, an electrical conductivity sensor, a soil sampler, or other sensors. The sensor electronics are electrically connected to the power source 310 and is configured to process the data collected from the various sensors. An additional body 320 may be connected to the main body 302 and may be configured to have a corresponding internal chamber 322. One or more of the elements 306, 308, 310, and 312 may be stored in this secondary internal chamber 322. A communication passage 324 may be formed between the main internal chamber 304 and the secondary internal chamber 322 so that one or more wires may extend from one chamber to the other. The secondary internal chamber 322 is also sealed from the ambient environment. The additional body 320 may be attached to a coupler 326 that can be connected to a cable (tether) 328 from a mother vessel, as discussed later. The tether may be used to move the device 300 up and down and also to transmit electrical power and data from the device to the mother vessel and vice versa.

The main body 302 is connected to a connector 314, which closes one end of the internal housing 304. One or more probes 330 are attached to the connector 314. A probe 330 may be configured as a tube that is connected to the connector 314 and is configured to collect a part of the ambient soil and/or water into which the device 300 is placed. Also connected to the connector 314 there is a sensing module 340 that is shaped as a tubular rod that extends past the connector 314 and the probes 330. The sensing module 340 is configured to receive one or more sensors 342 (only one shown for convenience). The sensor 342 can be one of temperature, pressure, electrical conductivity, chemical, biological, soil sampler, magnetic, radioactive, or any other sensor that is used when studying the ocean bottom. While the one or more sensors is located in the sensing module 340, part of the electronics supporting the sensor is located in the main or secondary bodies, as sensor electronics 312, as discussed above.

The sensing module 340 is designed to be reconfigurable so that any number of sensors, as desired by the operator of the device 300, can be added. In other words, the sensors can be added or removed from the sensing module as required by the operator. This means that the one or more sensors can be attached to or removed from the sensing module.

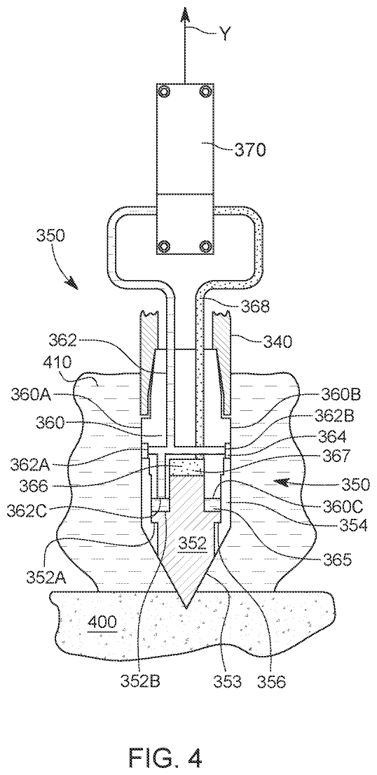

At the tip of the sensing module 340, there is a tip resistance module 350. The tip resistance module is illustrated in more detail in FIG. 4 and includes a tip 352 that is configured to have a pointed surface 353 that directly contacts the sediment 400 or ocean bottom to be measured. FIG. 4 shows the tip 352 being partially embedded into the sediment 400 and partially being surrounded by the ocean water 410. The pointed surface 353 may be inclined relative to the vertical axis Y with an angle between 5 and 65 degrees. An area of the pointed surface may be between 5 and 20 cm².

The tip resistance module 350 also includes a sleeve 354 that is configured to partially enclose the tip 352. The sleeve 354 has a shoulder 356 that is configured to engage a corresponding shoulder 352A of the tip 352, so that the two elements are mechanically connected and one does not slide relative to the other one when the entire device is driven into the sediment 400. The tip resistance module 350 further includes a core 360, which extends along the vertical axis Y and connects the sensing module 340 to the sleeve 354 and tip 352. The core 360 may be made of metal and includes a few passages for allowing water and oil to move freely between desired locations of the device 300, as discussed later. The sleeve 354 is fixedly attached to the core 360. However, the tip 352 is slidably attached to the sleeve 354, so that the tip 352 can move vertically up and down between the sleeve 354 and the core 360.

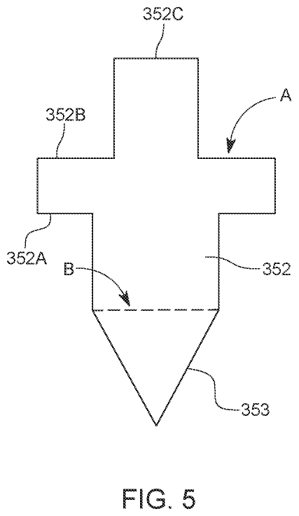

More specifically, as illustrated in FIG. 4, there is a first water passage 362 that connects the sides 360A and 360B of the core 360 to a bottom 360C of the core 360. The first water passage 362 terminates with ports 362A and 362B at the side walls 360A and 360B of the core 360. The first water passage 362 also communicates with a differential pressure sensor 370. The differential pressure sensor 370 can be located in the sensing module 340 or even in the main body 302. The first water passage 362 is provided with filters 364 on each of the sides 360A and 360B to prevent particulates from the sediment 400 to enter inside the core 360. The first water passage 362 has a third port 362C that fluidly communicates with an annular water chamber 365, formed between a bottom part of the core 360 and a top annular surface 352B of the tip 352. The various surfaces of the tip 352 are more clearly illustrated in FIG. 5. Note that the tip 352 also has a central top surface 352C, which faces an oil chamber 366, which is filled with oil 367, as illustrated in FIG. 4. The oil chamber 366 is defined by the central top surface 352C of the core 352, and side walls of the core 360, as illustrated in FIG. 4. The oil chamber 366 fluidly communicates with the second oil passage 368 and also with the differential pressure sensor 370.

In this way, the hydrostatic water pressure acts through port 362A and first water passage 362 on the top annular surface 352B of the tip 352, but also on the bottom pointed surface 353, reducing its effect on the tip 352. If the area A of the top annular surface 352B is sized to be equal with a horizontal projection area B of the bottom pointed surface 353, then the effect of the hydrostatic water pressure on the tip 352 is effectively cancelled. This means that the force exerted by the sediment 400 on the bottom pointed surface 353 is fully transmitted to the oil 367 in the oil chamber 366, and then the second oil channel 368 transmits this pressure to the differential pressure sensor 370. As the oil in the oil chamber 366 is also pressurized due to the hydrostatic pressure transmitted through the walls of the device and also due to the interaction between the tip 352 and the sediment 400, by subtracting in the differential pressure transducer 370 and thus the hydrostatic pressure, only the effect of the sediment on the tip can be measured, which is indicative of the penetration resistance.

Therefore, the device 300 discussed herein is capable to remove the hydrostatic water pressure from the measurement of the penetration resistance, which ensures that the small value of the penetration resistance is not obscured by the large value generated by the hydrostatic water pressure. Note that for this configuration, when the device 300 is lowered through the water toward the ocean bottom, but has not yet reached the ocean bottom, the pressures read by the differential pressure sensor 370, from the first water passage 362 and the second oil passage 368 are equal, which indicates that the hydrostatic water pressure is transmitted through the walls of the device to the oil in the oil chamber 366.

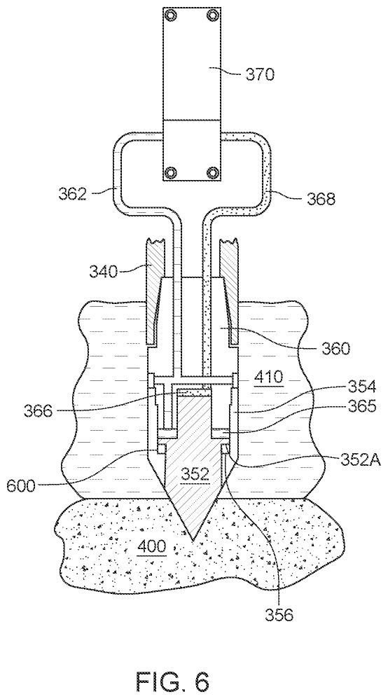

FIG. 6 shows the tip 352 being more embedded into the sediment 400 and less enclosed by the water 410. Because of the resistance exhibited by the sediment 400, the tip 352 has moved upward, toward the core 360, which resulted in the formation of an annular chamber 600 between the interior of the sleeve 356 and the lower side of the tip shoulder 352A. In addition, the upward movement of the tip 352 while the core 360 and the sleeve 354 remain fixed, resulted in the decrease in volume of the oil chamber 366 and also the decrease in volume of the annular water chamber 365, as also illustrated in FIG. 6.

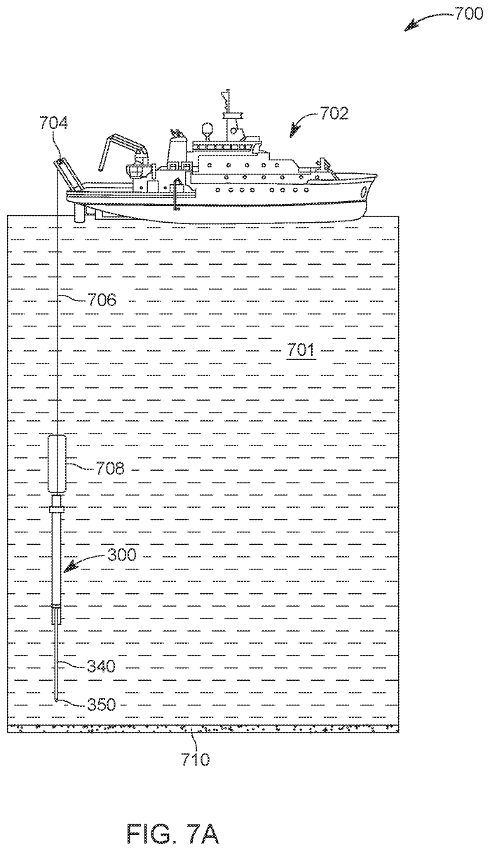

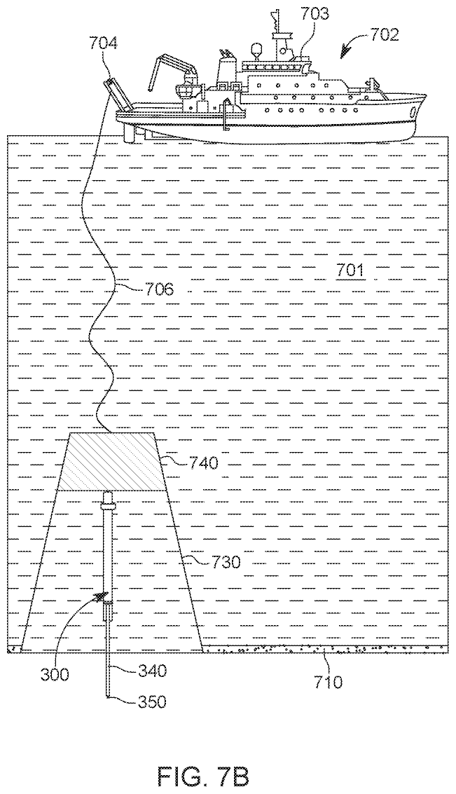

The device 300 can be deployed to the ocean bottom following various approaches. Two of these approaches are discussed with regard to FIGS. 7A and 7B. FIG. 7A shows a configuration 700 in which a mother vessel 702 takes the device 300 to a desired location relative to the ocean bottom. The vessel 702 has a winch 704 or equivalent device for metering a cable 706 into the water 701. Cable 706 may include a first element for providing resistance, and a second element for providing power and data exchange to the device 300. However, the second element is optional. A weight 708 is added to the cable 706 for driving the device 300 into the ocean bottom 710. The device 300 is shown in FIG. 7A approaching the ocean bottom 710. The sensing module 340 and the tip resistance module 350 are also visible in this figure.

A second approach for delivering the device 300 to the ocean bottom 710 is illustrated in FIG. 7B. In this configuration, a driving structure 730 is placed around the device 300 and is lowered to the ocean bottom 710 with the cable 706. Once on the ocean bottom 710, a driving mechanism 740 (for example, a moving weight or a piston) is activated by a controller 703 located on the vessel 702, for pushing the device 300 into the ocean bottom. As the device 300 is entering the sediment associated with the ocean bottom 710, the hydrostatic water pressure is fully balanced on the tip resistance module 350, and the differential pressure sensor 370 is capable to measure the pressure associated with the penetration resistance experienced by the tip of the resistance module 352. The data associated with these pressures is either stored in the local memory 308 of the device 300, for later analysis, or is transmitted in real time to the controller 703 of the vessel 702, through the cable 706. Additional data is also stored in the local memory or transmitted to the vessel, for example, data associated with the temperature of the water, pressure, electrical conductivity, radioactivity, pH, etc.

One skilled in the art will understand that the device 300 may also be deployed in a well, onshore or offshore, for determining the penetration resistance at the bottom of the well. For this application, the drilling tools would be taken out of the well, the device 300 will be lowered into the well based on one of the approaches discussed above, then the device will be pushed into the bottom of the well and measurements will be performed, after which the device is taken out and the drilling tool is lowered back into the well and the drilling is resumed. Based on the penetration resistance measurements, the type of drill used may be changed before the drilling resumes. Other applications of the device 300 may be envisioned, for example, in relation to marine and fluvial ports, oil and gas recovery, mining, seabed survey, etc.

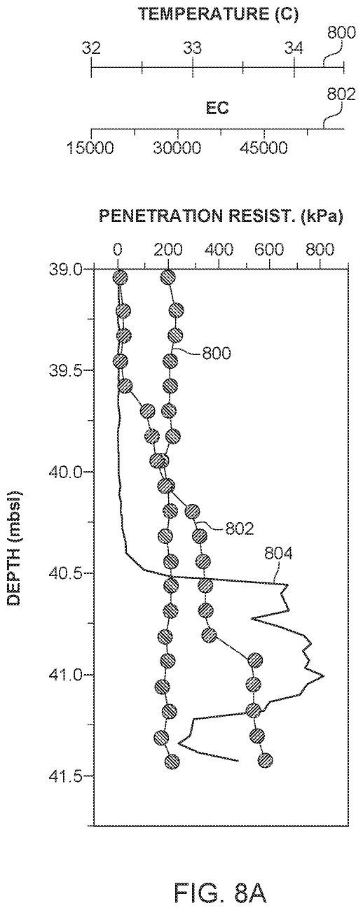

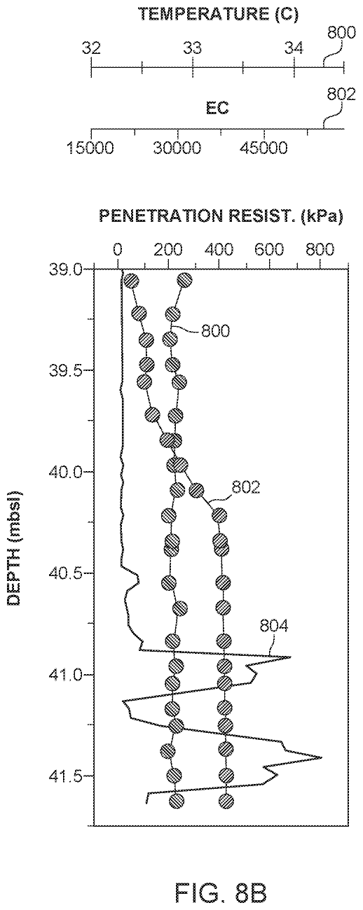

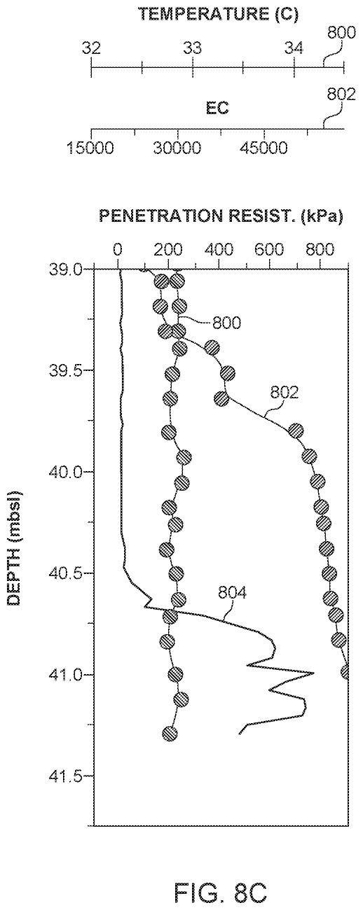

The temperature, electrical conductivity, and penetration resistance data was acquired with the device 300 for three different locations on the ocean floor and this data is illustrated in FIGS. 8A, 8B and 8C. Each figure plots the data for a given location and each figure shows the temperature 800, the electrical conductivity (EC) 802, and the penetration resistance 804. The axes for this data are shown on the top of each figure, as the X axes. The Y axis plots the depth of the tip 352 where the data is measured, where the depth is expressed in meters below sea level (mbsl). While the temperature at each location was substantially constant over approximately 3 meters of data collection, the EC and penetration resistance increased with the increase in depth, as expected.

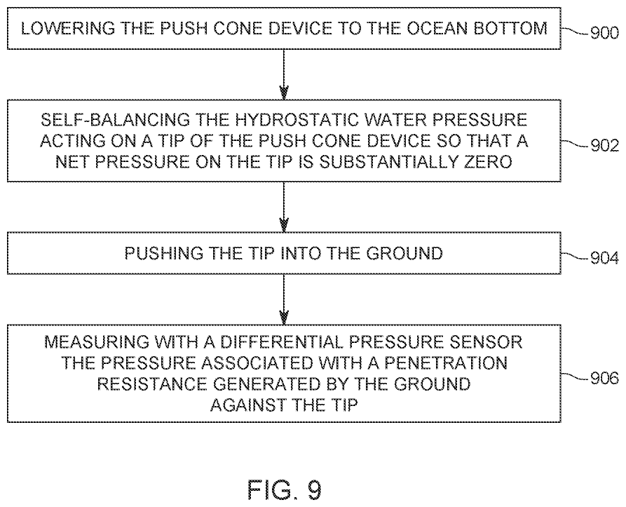

A method for collecting this data is now discussed with regard to FIG. 9. The method includes a step 900 of lowering the push cone device 300 to the ocean bottom, a step 902 of self-balancing the hydrostatic water pressure acting on a tip 352 of the push cone device 300 so that a net pressure on the tip 352 is negligible (the term negligible is understood in this application as meaning less than 10% of the current hydrostatic water pressure where the measurement is taking place), a step 904 of pushing the tip 352 into the ground, and a step 906 of measuring with a differential pressure sensor 370 the pressure associated with a penetration resistance generated by the ground against the tip. The differential pressure sensor 370 is configured to be in fluid communication, at a first port, with an oil chamber located inside the push cone device and, at a second port, with a water passage also inside the push cone.

The disclosed embodiments provide a hydrostatically-balanced push cone device for measuring a penetration resistance in the ocean bottom. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A push cone device for measuring a penetration resistance into ground, the device comprising:
a body having an internal chamber;
electronics located in the internal chamber;
a sensing module (attached to the body and configured to house one or more sensors;
a tip resistance module attached to the sensing module and having a tip that is configured to be fully hydrostatically-balanced under water; and
a differential pressure sensor that measures the penetration resistivity experienced by the tip resistance module.

2. The device of claim 1, wherein the tip resistance module comprises:
the tip;
a sleeve; and
a core.

3. The device of claim 2, wherein the sleeve is fixedly attached to the core and the tip is slidably attached to the sleeve.

4. The device of claim 2, wherein the core and the tip define an oil chamber that is filled with oil.

5. The device of claim 4, wherein the oil chamber is fluidly connected to a first port of the differential pressure sensor, which is located in the sensing module.

6. The device of claim 5, wherein the tip, the core and the sleeve form an annular water chamber.

7. The device of claim 6, wherein the annular water chamber freely communicates with an ambient of the device.

8. The device of claim 7, wherein the annular water chamber fluidly communicates with a second port of the differential pressure sensor.

9. The device of claim 8, wherein the differential pressure sensor outputs a pressure exerted by the ground on the tip, free of a hydrostatic water pressure.

10. The device of claim 8, wherein a water pressure exerted on an outside surface of the tip is equal to a water pressure exerted by the water in the water chamber so that the hydrostatic water pressure on the tip cancels out.

11. The device of claim 1, wherein the one or more sensors are modular and are removably attached to the sensing module.

12. A push cone device for measuring a penetration resistivity into ground, the device comprising:
a body having an internal chamber that houses electronics; and
a tip attached to the body and being configured to move relative to the body,
wherein the tip is configured to be pressure balanced under water.

13. The device of claim 12, further comprising:
a sensing module directly attached to the body;
a differential pressure sensor located in the sensing module and configured to measure the penetration resistivity; and
a tip resistance module directly attached to the sensing module,
wherein the tip resistance module includes the tip.

14. The device of claim 13, wherein the tip resistance module comprises:
a sleeve; and
a core,
wherein the sleeve is fixedly attached to the core and the tip is slidably attached to the sleeve.

15. The device of claim 14, wherein the core and the tip define an oil chamber that is filled with oil, the oil chamber is fluidly connected to a first port of the differential pressure sensor.

16. The device of claim 15, wherein the tip, the core, and the sleeve form an annular water chamber, and the annular water chamber freely communicates with an ambient of the device and the annular water chamber fluidly communicates with a second port of the differential pressure sensor.

17. The device of claim 16, wherein the differential pressure sensor outputs a pressure exerted by the ground on the tip, free from a hydrostatic water pressure.

18. The device of claim 16, wherein a water pressure exerted on an outside surface of the tip is equal to a water pressure exerted by the water in the water chamber so that the hydrostatic water pressure on the tip cancels out.

19. A method for measuring a penetration resistance with a push cone device, the method comprising:
lowering the push cone device to the ocean bottom;
self-balancing a hydrostatic water pressure acting on a tip of the push cone device so that a net pressure on the tip is negligible;
pushing the tip into the ground; and
measuring with a differential pressure sensor a pressure associated with the penetration resistance generated by the ground against the tip,
wherein the differential pressure sensor is configured to be in fluid communication, at a first port, with an oil chamber located inside the push cone device and, at a second port, with a water passage also inside the push cone.

20. The method of claim 19, wherein the oil chamber is filled with oil and is bordered only by the tip and a core.
